(19) United States
(12) Patent Application Publication
(10) Pub. No.: US 2004/0197821 A1
(43) Pub. Date: Oct. 7, 2004

(54) Rapid-detection biosensor

(76) Inventor: Alan Joseph Bauer, Jerusalem (IL)

Correspondence Address: Arthur S. Bickel, Biosensor Systems Design, Inc, 49 Ussishkin St., Jerusalem, 94542, IL

(21) Appl. No.: 10/407,690

(22) Filed: Apr. 4, 2003

(51) Int. Cl. G01N 33/53; G01N 33/542; C12M 1/34

(52) U.S. Cl. 435/7.1; 435/287.2

(57) ABSTRACT

A sensor for detecting analytes of interest is described. Analyte presence or concentration is determined through measurement of changes in an electrical property in a sensor circuit during analyte exposure to the sensor. The device immobilizes natural or synthetic macromolecules sufficiently close to a capacitor, so that binding of target analyte leads to charging of the capacitor. Current flow resulting from the capacitor charging is measured in an associated detection unit.

### BACKGROUND OF THE INVENTION

1. Field of the Invention

This invention pertains to a sensor and method for detecting or quantifying analytes. More particularly the present invention is directed to the detection of analytes by certain de novo electrical interactions thereof with an immobilized macromolecular binding agent and the analysis of effects that are produced as a result of such interactions.

2. Description of the Related Art

Chemical and biological sensors are devices that can detect or quantify analytes by virtue of interactions between targeted analytes and macromolecular binding agents such as enzymes, receptors, DNA strands, heavy metal chelators, or antibodies. Such sensors have practical applications in many areas of human endeavor. For example, biological and chemical sensors have potential utility in fields as diverse as blood glucose monitoring for diabetics, detection of pathogens commonly associated with spoiled or contaminated food, genetic screening, and environmental testing.

Chemical and biological sensors are commonly categorized according to two features, namely, the type of material utilized as binding agent and the means for detecting an interaction between binding agent and targeted analyte or analytes. Major classes of biosensors include enzyme (or catalytic) biosensors, immunosensors and DNA biosensors. Chemical sensors make use of synthetic macromolecules for detection of target analytes. Some common methods of detection are based on electron transfer, generation of chromophores, or fluorophores, changes in optical or acoustical properties, or alterations in electric properties when an electrical signal is applied to the sensing system.

Enzyme (or catalytic) biosensors utilize one or more enzyme types as the macromolecular binding agents and take advantage of the complementary shape of the selected enzyme and the targeted analyte. Enzymes are proteins that perform most of the catalytic work in biological systems and are known for highly specific catalysis. The shape and reactivity of a given enzyme limit its catalytic activity to a very small number of possible substrates. Enzymes are also known for speed, working at rates as high as 10,000 conversions per second per enzyme molecule. Enzyme biosensors rely on the specific chemical changes related to the enzyme/analyte interaction as the means for determining the presence of the targeted analyte. For example, upon interaction with an analyte, an enzyme may generate electrons, a colored chromophore or a change in pH (due to release of protons) as the result of the relevant catalytic enzymatic reaction. Alternatively, upon interaction with an analyte, an enzyme may cause a change in a fluorescent or chemiluminescent signal that can be recorded by an appropriate detection system.

Immunosensors utilize antibodies as binding agents. Antibodies are protein molecules that bind with specific foreign entities, called antigens, which can be associated with disease states. Antibodies attach to antigens and may remove the antigens from a host. Additionally or alternatively, the antibodies may trigger an immune response. Antibodies are quite specific in their interactions and, unlike enzymes, they are capable of recognizing and selectively binding to very large bodies such as single cells. Thus, antibody-based biosensors allow for the identification of certain pathogens such as dangerous bacterial strains. As antibodies generally do not perform catalytic reactions, there is a need for special methods to record the moment of interaction between target analyte and recognition agent antibody. Changes in mass (surface plasmon resonance, acoustic sensing) are often recorded; other systems rely on fluorescent probes that give signals responsive to interaction between antibody and antigen. Alternatively, an enzyme bound to an antibody can be used to deliver the signal through the generation of color or electrons; the enzyme-linked immunosorbent assay (ELISA) is based on such a methodology.

DNA biosensors utilize the complementary nature of the nucleic acid double-strands and are designed for the detection of DNA or RNA sequences usually associated with certain bacteria, viruses or given medical conditions. A sensor generally uses single-strands from a DNA double helix as the binding agent. The nucleic acid material in a given test sample is then denatured and exposed to the binding agent. If the strands in the test sample are complementary to the strands used as binding agent, the two interact. The interaction can be monitored by various means such as a change in mass at the sensor surface or the presence of a fluorescent or radioactive signal. Alternative arrangements provide binding of the sample of interest to the sensor and subsequent treatment with labeled nucleic acid probes to allow for identification of the sequences of interest.

Chemical sensors make use of non-biological macromolecules as binding agents. The binding agents show specificity to targeted analytes by virtue of the appropriate chemical functionalities in the macromolecules themselves. Typical applications include gas monitoring or heavy metal detection; the binding of analyte may change the conductivity of the sensor surface or lead to changes in charge that can be recorded by an appropriate field-effect transistor (FET). Several synthetic macromolecules have been used successfully for the selective chelation of heavy metals such as lead.

The present invention has applicability to all of the above noted binding agent classes.

Known methods of detecting interaction of analyte and binding agent can be grouped into several general categories: chemical, optical, acoustical, and electrical. In the last, a voltage or current is applied to the sensor surface or an associated medium. As binding events occur on the sensor surface, there are changes in electrical properties of the system. The leaving signal is altered as function of analyte presence.

The most relevant prior art to the present invention involves sensors that are based on electrical means for analyte detection. There are several classes of sensors that make use of applied electrical signals for determination of analyte presence. Amperometric sensors make use of oxidation-reduction chemistries in which electrons or electrochemically active species are generated or transferred due to analyte presence. An enzyme that interacts with an analyte may produce electrons that are delivered to an appropriate electrode; alternatively, an amperometric sensor may employ two or more enzyme species, one interacting with analyte, while the other generates electrons as a function of the action of the first enzyme, an arrangement known as a coupled enzyme system. Glucose oxidase has been used frequently in amperometric biosensors for glucose quantification for diabetics. Other amperometric sensors make use of electrochemically active species whose presence alters the system applied voltage as recorded at a given sensor electrode. Not all sensing systems can be adapted for electron generation or transfer, and thus many sensing needs cannot be met by amperometric methods alone. The general amperometric method makes use of an applied voltage and effects of electrochemically active species on said voltage. An example of an amperometric sensor is described in U.S. Pat. No. 5,593,852 to Heller, et al., which discloses a glucose sensor that relies on electron transfer effected by a redox enzyme and electrochemically-active enzyme cofactor species.

An additional class of electrical sensing systems includes those sensors that make use primarily of changes in an electrical response of the sensor as a function of analyte presence. Some systems pass an electric current through a given medium. If analyte is present, there is a corresponding change in an exit electrical signal, and this change implies that analyte is present. In some cases, the binding agent-analyte complex causes an altered signal, while in other systems, the bound analyte itself is the source of changed electrical response. Such sensors are distinguished from amperometric devices in that they do not necessarily require the transfer of electrons to an active electrode. Sensors based on the application of an electrical signal are not universal, in that they depend on alteration of voltage or current as a function of analyte presence; not all sensing systems can meet such a requirement. An example of this class of sensors is U.S. Pat. No. 5,698,089 to Lewis, et al., which discloses a chemical sensor in which analyte detection is determined by a change of an applied electrical signal. Binding of analyte to chemical moieties arranged in an array alters the conductivity of the array points; unique analytes can be determined by the overall changes in conductivity of all of the array points. The present invention does not rely on arrays or changes of applied electrical signal as a function of analyte presence. The present sensor does not require any applied electrical or electromagnetic signal.

Several other publications that do not fall into the preceding categories are worthy of mention in the prior art. The document, Direct Observation of Enzyme Activity with the Atomic Force Microscope. Radmacher, Manfred et al. Science. 265:1577, 9 Sep. 1994 noted the existence of augmented spatial fluctuations in enzymes interacting with substrates, but did not apply this phenomenon to analyte detection.

U.S. Pat. No. 5,620,854 to Holzrichter, et al., pro posed the use of macromolecule motion to detect analyte. The disclosed system relies specifically on atomic force or scanning tunneling microscopes for detection of said motion.

U.S. Pat. No. 5,114,674 to Stanbro, et al. discloses a sensor that is based on the interference of applied electrical fields. Interaction of target analyte with a binding agent alters the interference of the applied electrical field.

Other prior-art voltage-based sensors require the use of semiconducting field-effect transistors and rely on the chemical generation or physical trapping of charged species near the sensor surface. This approach has found widespread use in the detection of positively-charged heavy metals as well as analytes that are involved in proton (H+) generating enzyme reactions. The document Endoscopic Urease Sensor System for Detecting Helicobacter pylori on Gastric Mucosa, Sato et al. Gastrointestinal Endoscopy 49:32-38 (1999) describes a pH-sensitive FET for the detection of the enzyme urease, associated with the pathogenic bacterium H. pylori.

While hundreds of sensors have been described in patents and in the scientific literature, actual commercial use of such sensors remains limited. In particular, virtually all sensor designs set forth in the prior art contain one or more inherent weaknesses. Some lack the sensitivity and/or speed of detection necessary to accomplish certain tasks. Other sensors lack long-term stability. Still others cannot be sufficiently miniaturized to be commercially viable or are prohibitively expensive to produce. Some sensors must be pre-treated with salts and/or enzyme cofactors, a practice that is inefficient and bothersome. To date, virtually all sensors are limited by the known methods of determining that contact has occurred between an immobilized binding agent and targeted analytes. Use of fluorescent or other external detection probes adds to sensor production requirements and reduces lifetimes of such sensor systems. Additionally, the inventor believes that there is no sensor method disclosed in the prior art that is generally applicable to the vast majority of macromolecular binding agents, including enzymes, antibodies, antigens, nucleic acids, receptors, and synthetic binding agents.

### SUMMARY OF THE INVENTION

It is therefore a primary object of some aspects of the present invention to provide an improved analyte detection system, in which a detection unit is electrically connected to a sensor strip so as to allow for detection of de novo electrical currents in a sensor circuit that are responsive to analyte presence.

It is a further object of some aspects of the invention to describe an electrical circuit that includes a capacitor-based sensor strip for sensitive and inexpensive analyte detection.

It is an additional object of some aspects of the invention to improve the consistency and ease of use in detection of an analyte in a sensor system by inclusion of a dielectric material between first and second conductive elements.

In contrast to the above noted U.S. Pat. No. 5,593,852, the practice of the present invention does not require application of an external voltage, oxidation-reduction chemistry, or exogenous electron generation or transfer. Furthermore, in contrast to the above noted disclosures, the present invention does not rely on arrays or changes of applied electrical fields or signals as a function of analyte presence.

The invention is an extension of the sensor and method described in PCT application PCT/US00/15400 of common assignee herewith, and herein incorporated by reference. The sensor disclosed in PCT application PCT/US00/15400 is based on detection of de novo electrical signals, and is capable of rapid determination of analyte presence in complex sample matrices. Structural changes involving components of the sensor circuit disclosed herein provide for further improved analyte detection through detection and monitoring of phenomena, including electrical signals that are generated in a sensor circuit during analyte interaction.

As described in the noted PCT application PCT/US00/15400, which discloses a sensor circuit incorporating a base member, or first conducting element and a binding agent layer associated with the first conducting element. As disclosed in the noted PCT application, the methodology of analyte detection is very sensitive. Using the improvements of the present invention, it is possible to detect specific pathogenic bacteria consistently in a complex meat matrix within two minutes at 1-10 cells per milliliter of sample. In general, measurement of de novo current in a sensor circuit according to the present invention allows for rapid, specific and sensitive determination of analyte presence.

A sensor strip according to the invention may contain a plurality of identical or unique sensor strips so as to increase system detection redundancy or multiple analyte detection capabilities. Component strips of a composite sensor strip may be individually monitored, each component strip forming a part of a different sensor circuit.

In preferred embodiments of the invention sensor strips are unpowered, that is, no external electrical signal is applied to them. In other preferred embodiments, the sensor strip may be powered through application of voltage, current, or other electrical signal to the sensor strip. In some embodiments, a plurality of sensor strips may be employed in the detection of one or a plurality of analytes.

Contact with the sensor strip is generally electrically passive in nature and occurs at one or two positions. One of the contacting electrodes may serve as an electron sink or electrical ground. The electrodes may be prepared from either conducting or semiconducting materials or a combination thereof. The electrodes are generally equipotential. In preferred embodiments employing electrically passive electrode contact with the sensor strip, neither electrode is used to deliver an external electrical signal to the unpowered sensor strip. The two electrodes associated with each sensor strip may be prepared from the same or different materials.

A detection unit is generally contacted to a sensor strip at two positions through passive contact of associated equipotential electrodes and the detection unit generally measures de novo current flow or voltage in a closed circuit. The detection unit may simultaneously measure more than one type of signal and it may be contacted to a plurality of sensor strips. Current measurement may be direct or over a resistor for a voltage reading. A reading or other indication is recorded when a generated current is passed over a voltmeter resistor to yield a value read as a voltage, though the original signal is a de novo current responsive to analyte presence. Additionally, the detection unit may further process the signal or a component thereof for the purpose of analyte detection and concentration range determination. In some preferred embodiments, a detection unit is unnecessary, as the generated current leads directly or otherwise to electroluminescence.

The invention provides a sensor for detecting an analyte, which includes a base member or first conductive element, a binding agent layer proximate the base member, a dielectric element proximate the base member, and a second conductive element that is physically contacted to the dielectric element and adapted for electrical connection to the base member. The base member and the binding agent layer minimally define a sensor strip, while additional layers such as the dielectric element or the second conductive element may be included in the term “sensor strip” if they are physically associated with the base member when the base member is not contacted with the detection unit. The first and second conductive elements surround the dielectric element and form a structure similar to that of an electrical capacitor.

An aspect of the sensor includes a chemical entity bound to the base member and disposed proximate the binding agent layer.

Yet another aspect of the sensor includes two equipotential leads coupling the sensor strip to a detection unit, wherein at least one of the equipotential leads is electrically contacted to the second conductive element.

According to an additional aspect of the sensor, the second conductive element is an element of an electrode of the detection unit. The second conductive element is brought into contact with the dielectric element.

One aspect of the sensor includes a packaging layer disposed above the binding agent layer. The packaging layer is soluble in a medium that contains the analyte.

According to another aspect of the sensor, the dielectric element is an organic polymer and is physically associated with the base member on a first side of the base member, and the binding agent layer is immobilized on one or both sides of the base member.

According to a further aspect of the sensor, the sensor strip includes a plurality of sensor strips.

The invention provides a method for detecting a predetermined analyte, including the steps of providing an electrically conductive base member, and forming a binding agent layer of macromolecules in proximity to the base member, wherein the macromolecules are capable of interacting at a level of specificity with the predetermined analyte. The method further includes disposing a dielectric element proximate the base member, wherein the base member, the binding agent layer and the dielectric element minimally define a sensor strip, disposing a second conductive element proximate the dielectric element, exposing the predetermined analyte to the binding agent layer, and, detecting an electrical current generated in a closed electrical circuit. The current is responsive to presence of the predetermined analyte. The closed electrical circuit minimally includes the first conductive element, the dielectric element and the second conductive element as well as a detection unit connectable thereto.

An aspect of the method includes binding a chemical entity to the base member, and forming the binding agent layer proximate the chemical entity.

In another aspect of the method, detecting is performed by equipotentially coupling leads of a detection unit to the sensor strip, wherein one of the leads is coupled to the second conductive element.

According to an additional aspect of the method, the dielectric element is an organic compound, and is physically associated with the base member on one side of the base member. The binding agent layer is immobilized on one or more sides of the base member.

One aspect of the method includes disposing a packaging layer above the binding agent layer. The packaging layer is soluble in a medium that contains the predetermined analyte.

According to another aspect of the method, the sensor strip includes a plurality of sensor strips.

### BRIEF DESCRIPTION OF THE DRAWINGS

For a better understanding of these and other objectives of the present invention, reference is made to the following detailed description of the invention, by way of example, which is to be read in conjunction with the following drawings, wherein:

FIG. 1 is a schematic view of a sensor detection system, which is constructed and operative in accordance with a preferred embodiment of the invention, wherein a sensor strip comprised of a base member, chemical entity, binding agent layer and packaging layer forms a closed sensor circuit with electrodes, a dielectric element, a second conductive element and a detection unit;

FIG. 2 is a plot of data from a control experiment, using the system shown in FIG. 1, in which the binding agent was a monoclonal antibody for pathogen, E. coli 0157:H7;

FIG. 3 is a plot of data from an experiment performed under the conditions of the experiment shown in FIG. 2, in which target analyte was present;

FIG. 4 is a schematic view of a sensor detection system, which is constructed and operative in accordance with an alternative embodiment of the invention, wherein a dielectric element is associated with a sensor strip;

FIG. 5 is a schematic view of a sensor detection system, which is constructed and operative in accordance with an alternative embodiment of the invention, wherein a dielectric element is placed between a base member and a second conductive element to form an electric capacitor;

FIG. 6 is a schematic view of a multiplexed alternative embodiment of a sensor detection system, which is constructed and operative in accordance with an alternate embodiment of the invention; and

FIG. 7 is a schematic view of sensor system, which is constructed and operative in accordance with an alternate embodiment of the invention showing a sensor strip in contact with a sample.

### DESCRIPTION OF THE PREFERRED EMBODIMENT

In the following description, numerous specific details are set forth in order to provide a thorough understanding of the present invention. It will be apparent, however, to one skilled in the art that the present invention may be practiced without these specific details. In other instances, well-known circuits and control logic have not been shown in detail in order not to unnecessarily obscure the present invention.

### Definitions

Certain terms are now defined in order to facilitate better understanding of the present invention.

An “analyte” is a material that is the subject of detection or quantification.

A “base member” is a solid or liquid element on or near which macromolecules can be physically or chemically immobilized for the purpose of analyte detection.

“Macromolecules”, “macromolecular binding agents”, “binding agents” or “macromolecular entities” can be any natural, mutated, synthetic, or semi-synthetic molecules that are capable of interacting with a predetermined analyte or group of analytes at a level of specificity.

A “binding agent layer” is a layer proximate the base member and composed of one or a plurality of binding agents. The binding agent layer may be composed of more than one type of binding agent. A binding agent layer may additionally include molecules other than binding agents. Cross-linking agents may be applied to bind separate components of a binding agent layer together.

A “chemical entity” is a chemical layer that is disposed proximate the base member on either one or both sides of the base member. It may serve to partially insulate the base member from direct contact with binding agents, or it may serve as the dielectric element defined below. Chemical entities may be differentially deposited on opposite sides of a base member by any means or multiple layers on a given side of the base member may be considered a single chemical entity. Natural oxides may serve the role of chemical entity.

A “packaging layer” is defined as a chemical layer disposed above the binding agent layer. The packaging layer may aid in long term stability of the macromolecules, and in the presence of a sample that may contain analyte of interest, the packaging layer may dissolve to allow for rapid interaction of analyte and binding agents. The packaging layer may also serve in conjunction with the charged macromolecules in the role of a dielectric element defined below. Such may be the case when a sensor is coated equally on both sides with chemical entities, macromolecules, and packaging layer.

A “sensor strip” is defined as a minimum of a single base member and its associated binding agent layer. If multiple macromolecular entities, chemical entities, packaging layers or other elements are physically associated with the base member, then they are included in the term “sensor strip”.

An “electrode” or “lead” is a wire, electrical lead, connection, electrical contact or the like that is attached at one end to a detection unit and contacted at the other end directly or indirectly to a sensor strip.

The terms “generated” and “de novo” electrical signals are used with respect to the electrical arts. Specifically by these terms, it is intended to exclude obligate oxidation-reduction chemistries and electrical phenomena resulting directly or otherwise from the necessary application of an external electrical or electromagnetic signal to sensor strip or sample. A generated or de novo electrical signal in the present invention is one that is produced in a sensor circuit as described herewith without any required application to the sensor strip of electrical or electromagnetic signal. Additionally, there is no oxidative transfer of electrons between the base member and binding agent, analyte, or sample.

A “detection unit” is any device or material that allows for the detection of one or more electrical signals generated in a sensor circuit.

“Dielectric element” refers to a material of an insulating property. The material may also show polarizability in the presence of an electric field. The dielectric element is included in a “sensor circuit” that minimally includes one such element in addition to a binding agent layer, a base member or first conductive element, and a second conductive element. The dielectric element may be present as a film, coating, solid element, or monolayer. A dielectric element is disposed between the first and second conductive elements and serves as a dielectric between the two conductive elements, as a dielectric does in a parallel-plate capacitor.

A “second conductive element” is an electrically-conducting material that is physically proximate the dielectric element and is distinct from the conductive base member. The second conductive element is electrically contacted to the base member, either directly or through the components of a sensor circuit. Coatings, foils, chips or pieces of metals such as gold, silver, aluminum, and copper are preferred in the role of second conductive element.

Without being bound by any particular theory, the following discussion is offered to facilitate understanding of the invention. The sensor design disclosed herein is based on de novo electrical signals generated in a sensor circuit as a function of analyte presence. The sensor utilizes a novel method of detecting an analyte wherein macromolecular binding agents are first immobilized as a binding agent layer proximate an electrically conductive base member. De novo electrical signals such as current in a circuit that includes the base member can be monitored for change during exposure of the macromolecular binding agents to a sample that may contain target analyte. In the present invention, the advantages of particular forms of sensor strip contact are disclosed more fully. Specifically, a dielectric element placed between base member and a second conductive element may be utilized in order to facilitate signal measurement and analyte detection through the analyte-related charging or discharging of a capacitor formed from the first and second conductive elements and the dielectric element physically placed between them. This capacitor may form an RC circuit with a resistor associated with a detection unit or a resistor added separately to a detection circuit. The specific capacitance value for the capacitor may vary over a wide range from femtofarad to microfarad values. As the goal in biosensing is rapid analyte detection and not full capacitor charging, picofarad to microfarad system capacitance, in combination with a resistance of about 100 ohms leads to an appropriately rapid current flow for practical analyte detection. These values are not critical. However, optima are application dependent. In current prototypes, a small circuit resistance is used, in which case it is recommended that the capacitance not be too large, due to a potential loss in sensitivity for analyte detection. When too small a value of capacitance is chosen, spurious background signals may interfere with proper analyte identification.

In the various embodiments disclosed herein, like elements have like reference numerals differing by multiples of 100.

### First Embodiment.

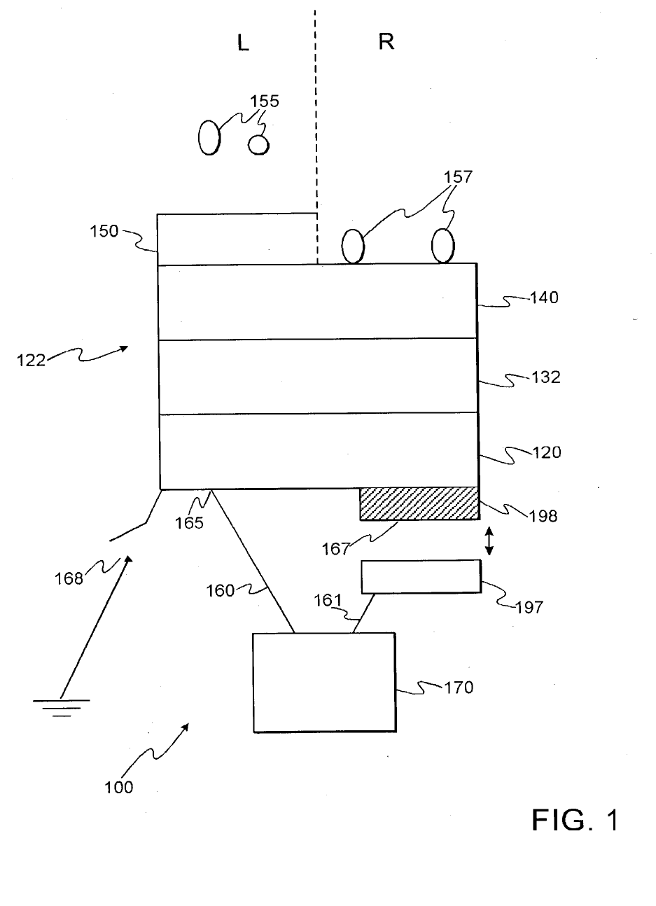

Reference is now made to FIG. 1, which is a schematic of a sensor detection system 100 that is constructed and operative in accordance with a preferred embodiment of the invention. The sensor detection system 100 comprises a sensor strip 122, which is part of a sensor circuit 120, 160, 170, 161, 197, 198 in which one or more electrical signals are generated internally in the sensor circuit 120, 160, 170, 161, 197, 198 itself. Provision is made for an external detection unit 170 to be coupled to the sensor strip 122 using equipotential, electrically-passive electrodes 160, 161 to provide contact between the sensor strip 122 and the detection unit 170. The equipotential passive electrodes 160, 161 of the detection unit 170 are contacted to the sensor strip 122 at a contact position 165 and to a dielectric element 198 at a contact position 167. The electrode 161 is provided with a second conductive element 197 in the form of a gold coating. In FIG. 1, the electrode 161 and the second conductive element 197 are shown in a non-contacting relationship with the sensor strip 122 for clarity of presentation, it being understood that in operation the second conductive element 197 is contacted with the dielectric element 198, as indicated by the double-headed arrow.

The sensor circuit 120, 160, 170, 161, 197, 198 models a metal-dielectric-metal capacitor, and has a resistance of approximately 100 ohms. The purpose of the dielectric element 198 is to aid in facile signal capture. The dielectric element 198 may be present as a coating, chip or other form. The presence of at least one dielectric element 198 between a base member 120 and the second conductive element 197 facilitates measurement of the capacitor 120, 198, 197 in the sensor circuit 120, 160, 170, 161, 197, 198. Interaction of analyte 155, 157 with a binding agent layer 140 draws electrons from the second conductive element 197 through the detection unit and associated leads 160, 161, 170 and into the base member 120, which is the first conductive element. The effect is to “charge” the capacitor represented by base member 120, dielectric element 198 and second conductive element 197. This charging effect is recorded as a generated current measured in the detection unit 170.

In general, metals are preferred in the roles of base member 120 and second conductive element 197. Conducting foils, coatings, thin-films, inks, and solid pieces are particularly preferred for the base member 120 and second conductive element 197.

The dielectric element 198 is preferably prepared from organic compounds, metal oxides or thin layers of insulators and is physically placed between the base member 120 and the second conductive element 197. Examples of appropriate dielectric elements include, but are not limited to mica, insulating coatings, electrolytes, ceramics, organic polymers such as polystyrene, polyethylene, polypropylene, Teflon®, polyvinyl chloride and the like. Dielectric elements may be incorporated directly into detection unit, associated electrodes or sensor strips. Preferred embodiments have both the dielectric layer 198 and the second conductive layer 197 physically associated with the sensor strip composed of base member 120 and binding agent layer 140.

Dielectric elements may be incorporated directly into detection unit, associated electrodes or sensor strips and are shown as a distinct elements in the accompanying figures for the purpose of convenience of presentation.

In particular applications, dimensions and spacing of the base member 120, the dielectric element 198, and the second conductive element 197 are selected for optimal delivery of electrons from the second conductive element 197 to the base member 120 by way of the detection unit 170.

The detection unit 170 may then measure a current, or other electrical signal generated in the sensor circuit 120, 160, 170, 161, 197, 198 as a function of analyte interaction with the sensor strip, as is disclosed in further detail hereinbelow.

The detection unit 170 may also serve to ground the sensor strip 122 prior to measurement, so that stray signals are removed prior to exposure of sample to the sensor strip 122. Such grounding may be performed either through an optional switched grounding electrode 168 or using a separate contact between the detection unit 170 and the sensor strip 122 (not shown). Grounding may also be performed at times during operation of the sensor detection system 100 in order to enhance signal quality.

The binding agent layer 140 is located proximate the base member 120. A chemical entity 132 is disposed between the base member 120 and the binding agent layer 140. Self-assembled monolayers are particularly preferred in the role of the chemical entity 132. Typically, the chemical entity 132 is a self-assembled monolayer (“SAM”) formed proximate the base member, with binding agent layer 140 disposed above the SAM. For the purposes of this invention, “proximate” with respect to the binding agent layer 140 disposition relative the base member is defined as any distance that allows for analyte-responsive generation of a de novo electrical signal in the sensor circuit 120, 160, 170, 161, 197, 198 as defined hereinabove.

An optional packaging layer 150, shown on the left side of FIG. 1, is a layer of water-soluble chemicals deposited above the immobilized macromolecules of the binding agent layer 140. The packaging layer 150 may be deposited by soaking or spraying methods. The packaging layer 150 serves to stabilize the binding agent layer 140 during prolonged storage. In the absence of a packaging layer, oil and dirt may build up on the hydrophilic binding agent layer 140 and may interfere with the rapid action of the sensor system. Glucose and a salt, such as sodium chloride, are typically used for the packaging layer 150 so as to guarantee their dissolution in aqueous samples, and thus facilitate direct interaction between macromolecular binding agents of binding agent layer 140 and analytes 157. Other chemicals may be chosen for use in the packaging layer. Water-soluble polymers, sugars, salts, organic, and inorganic compounds are all appropriate for use in preparation of the packaging layer 150.

As shown on the left side of FIG. 1, free analyte 155 is disposed proximate the packaging layer 150 prior to its dissolution. When the packaging layer 150 dissolves, the macromolecules incorporated in the binding agent layer 140 are free to immediately interact with analyte 157, as shown on the right side of FIG. 1. After dissolution of the packaging layer 150, analyte 157 is shown interacting with the binding agent layer 140 on the right side of FIG. 1. The analyte 155, 157 can be a member of any of the following categories, listed herein without limitation: cells, organic compounds, antibodies, antigens, virus particles, pathogenic bacteria, metals, metal complexes, ions, spores, yeasts, molds, cellular metabolites, enzyme inhibitors, receptor ligands, nerve agents, peptides, proteins, fatty acids, steroids, hormones, narcotic agents, synthetic molecules, medications, enzymes, nucleic acid single-stranded or double-stranded polymers. The analyte 155 can be present in a solid, liquid, gas or aerosol. The analyte 155 could even be a group of different analytes, that is, a collection of distinct molecules, macromolecules, ions, organic compounds, viruses, spores, cells or the like that are the subject of detection or quantification. Some of the analyte 157 physically interacts with the sensor strip 122 after dissolution of the packaging layer 150 and causes an increase in electrical signals generated in the sensor circuit 120, 160, 170, 161, 197, 198. Contact of electrodes 160, 161 to sensor strip 122 allows for measurement of such a de novo electrical signal that is responsive to analyte presence. There is no requirement for application of a voltage or other electrical signal to the sensor strip 122 prior to or during measurement of generated electrical signals by the detection unit 170. In some embodiments, one may apply such an external signal, in which case the generated electrical signal in the sensor system that is responsive to analyte presence will alter the exit signal.

Examples of macromolecular binding agents suitable for use as the binding agent layer 140 include, but are not limited to enzymes that recognize substrates and inhibitors, antibodies that bind antigens, antigens that recognize target antibodies, receptors that bind ligands, ligands that bind receptors, nucleic acid single-strand polymers that can bind to form DNA-DNA, RNA-RNA, or DNA-RNA double strands, and synthetic molecules that interact with targeted analytes. The present invention can thus make use of enzymes, peptides, proteins, antibodies, antigens, catalytic antibodies, fatty acids, receptors, receptor ligands, nucleic acid strands, as well as synthetic macromolecules in the role of the binding agent layer 140. Natural, synthetic, semi-synthetic, over-expressed and genetically-altered macromolecules may be employed as binding agents. The binding agent layer 140 may form monolayers, multilayers or mixed layers of several distinct binding agents or binding agents with other chemical components (not shown). A monolayer of mixed binding agents may also be employed (not shown). The binding agents in the binding agent layer 140 may be cross-linked together with glutaraldehyde or other chemical cross-linking agents.

The macromolecule component of the binding agent layer 140 is neither limited in type nor number. Enzymes, peptides, receptors, receptor ligands, antibodies, catalytic antibodies, antigens, cells, fatty acids, synthetic molecules, and nucleic acids are possible macromolecular binding agents in the present invention. The sensor detection system 100 may be applied to detection of many classes of analyte because it relies on the following-properties shared by substantially all applications and embodiments of the sensor detection system according to the present invention:

(1) that the macromolecules chosen as binding agents are highly specific entities designed to bind only with a selected analyte or group of analytes;

(2) that analytes have associated electrostatic fields;

(3) that binding of analyte electrostatically induces electrons to move between the second conductive layer and the base member; and,

(4) that this motion of electrons between elements of the detection circuit can be detected as an electrical current or other electrical signal in an associated detection unit.

The broad and generally applicable function of the sensor detection system 100 is preserved during formation of the binding agent layer 140 in proximity to the base member 120 because the binding agent layer 140 formation can be effected by either specific covalent attachment or general physical absorption. It is to be emphasized that the change in de novo signal that is associated with analyte presence does not depend on any specific enzyme chemistries, optical effects, fluorescence, chemiluminescence, oxidation-reduction phenomena or applied electrical signals. Additionally, there are no reference electrodes, and the two detection unit electrodes are generally equipotential prior to measurement of signal generated in the sensor circuit. These features are important advantages of the present invention.

The detection unit 170 is any device or material that can detect one or more de novo signals in a sensor circuit as a result of sensor strip exposure to a sample that contains analyte 155. Examples of such signals include but are not limited to electrical current; magnetic field strength; induced electromotive force; voltage; light; impedance; signal sign; frequency component or noise signature of a predetermined electrical signal propagated into a sensor strip at a first location and received at a second location. While the detection unit 170 may be a digital electrical metering device, it may also have additional functions that include, but are not limited to sensor strip grounding, data storage, data transfer, data processing, alert signaling, command and control functions, and process control. Detection units may be contacted through “leads”, realized as electrodes to one or a plurality of sensor strips. The detection unit 170 may be a digital voltmeter. In any case, the de novo signal produces a reading or indication in the detection unit 170. In some embodiments, the de novo signal may be an electrical voltage or a current, and the reading or indication can be a voltage value measured over an internal resistor of the detection unit 170.

Baseline readings in the detection unit 170 may be determined from a sample that lacks target analyte or analytes or by grounding the sensor strip 122 prior to sample exposure in a manner disclosed above.

The specific design of the detection unit 170 depends on what quantity or quantities are being observed, e.g., current, magnetic field flux, frequency, impedance. The detection unit may be integrated into a computer (not shown) or other solid-state electronic device for easier signal processing and data storage. The same or a different computer may be used to control sample application or sample serial dilution in order to monitor both sample manipulation as well as the generated electrical responses in a single or multiplexed sensor strip arrangement. The detection unit may also be a voltage-sensitive dye or colored material.

The implications of the analyte detection methodology are significant. Firstly, detection can take away from the direct point of macromolecule-analyte contact, as the electron flow can occur at a point removed from analyte-macromolecule interaction. This fact allows for closed-package “food sensing” or the sensing of potentially hazardous samples, e.g., blood in closed containers. One portion of the sensor contacts the material of interest, while detection of analyte-responsive de novo electrical signals occurs between on the exposed portion of the sensor strip.

The implications are that nearly any material that can be recognized at a level of specificity by a peptide, protein, antibody, enzyme, receptor, nucleic acid single strand, synthetic binding agent, or the like can be detected and quantified safely in food, body fluids, air or other samples quickly, cheaply, and with high sensitivity. Response is very rapid, generally less than 90 seconds. Cost of manufacture is low, and sensitivity has been shown to be very high.

### EXAMPLE 1

The analysis in this example was performed using the embodiment of FIG. 1. Ground turkey meat (5.11 g) was re-suspended in deionized water (40 ml). The suspension was vortexed and used as a background for detecting a specific bacterial strain. Sensor strips specific for pathogen E. coli 0157:H7 were prepared as follows. Aluminum foil having a matte surface and a shiny surface (Diamond Foil, Reynolds Metals Co., 555 Guthridge Court, Norcross, Ga. 30092) was treated with an aqueous solution of monoclonal antibody specific for E. coli 0157 (Product C65310M, Biodesign International, 60 Industrial Park Road, Saco, Me. 04072 USA) at an approximate concentration of 18 microgram per milliliter. The solution was at near pH 5.0, so as to increase the number of protonated carboxylic acid moieties on the protein for interaction with the aluminum oxide surface. The solution was kept in contact with the aluminum foil for approximately 20 minutes and then the aluminum foil was rinsed with deionized water. The aluminum was next rinsed with a concentrated solution of sodium chloride and sucrose and then allowed to air dry. In this example, the aluminum foil was used for the base member 120, the monoclonal antibodies formed the binding agent layer 140, and sodium chloride and sucrose made up the packaging layer 150. In this example, the natural aluminum oxide serves as chemical entity 132. While the antibodies were applied to the shiny side of the aluminum foil, an organic dielectric material was applied to the matte side, specifically opposite the location of the bound binding agent layer. Phthalate-containing commercial nail polish (Product No. 53 from A. Atar, Israel) was used as the dielectric element 198. Another suitable nail polish is Orly® Nail Color, Orly International, 9309 Deering Avenue, Chatsworth, Calif. 91311-5856, USA). It is believed that a dibutyl-phthalate component in the nail polish acts as an organic dielectric capable of separating the two plates of the capacitor, namely the base member 120 and the second conductive element 197. The polish was allowed to dry and strips were cut with approximate dimensions of 1 cm×4 cm. Individual strips were placed partially in an Eppendorf tube with the nail polish-treated side of the aluminum foil exposed for contact with electrodes attached to a Fluke 189 multimeter, having data collection software, which was used for the detection unit 170. Gold coated black and red banana leads of the Fluke Model 189 multimeter were used as the electrode 161 and the electrode 160 respectively. The black banana lead was contacted to the nail polish-treated surface, while the red banana lead was contacted directly to the aluminum foil. A gold coating on the black banana lead served as the second conductive element 197.

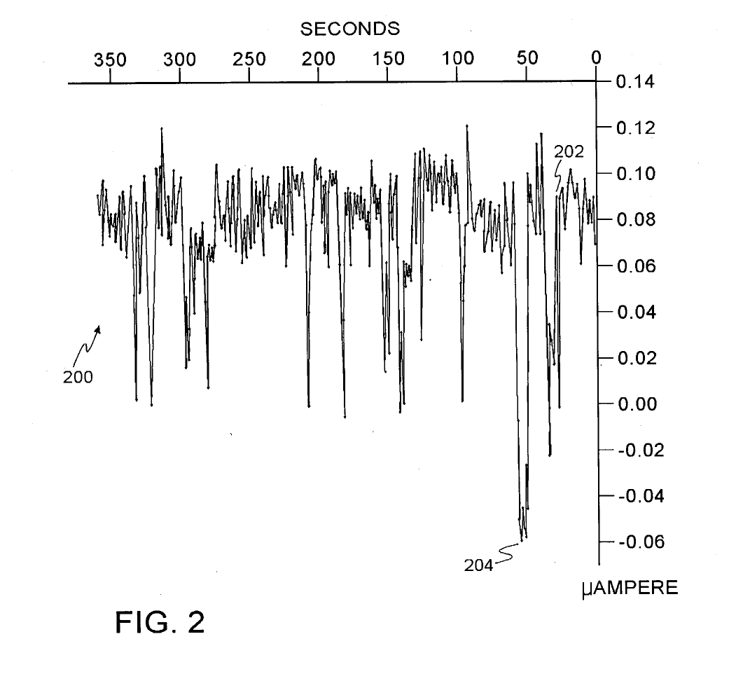

Reference is now made to FIG. 2, which is a signal time plot of the output of the Fluke Model 189 multimeter taken during exposure of a sensor strip, prepared according to this example, to the turkey-water suspension as a background experiment. As shown in a plot 200, there was no significant signal produced. When gold coating of the black banana lead contacted the dielectric element, at a point 202, rectified signal current produced a negative signal. The lowest reading recorded over an interval of six minutes was −0.06 microamperes, as indicated by a point 204. This sample was shown by plating and standard bacteriological culture to contain non-target bacteria, and not to contain the target bacterium, E. coli 0157:H7.

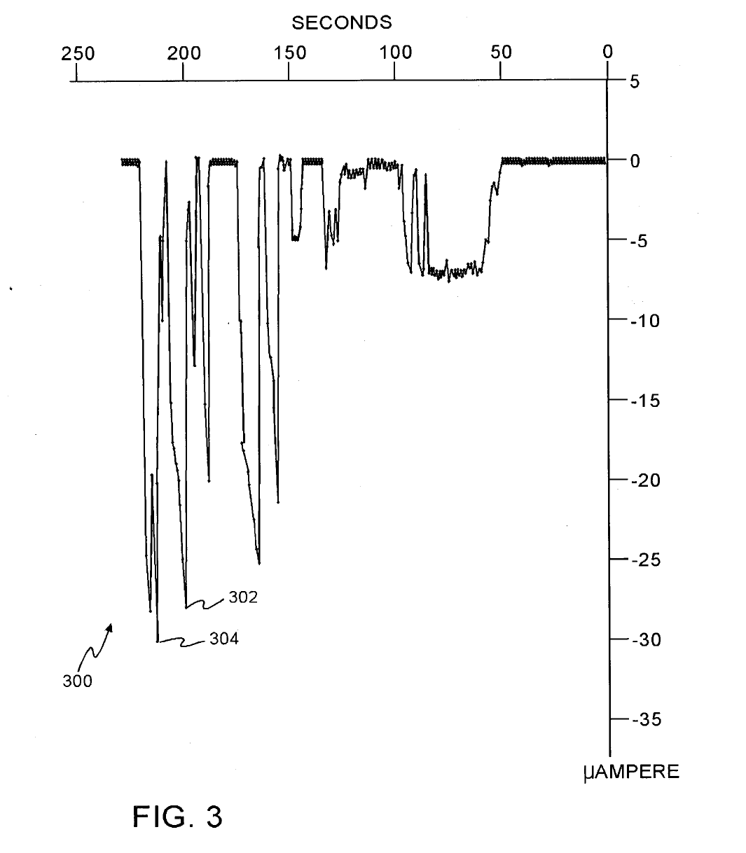

Reference is now made to FIG. 3, which is a signal time plot of the output of the Fluke 189 multimeter. A plot 300 was taken during exposure of another sensor strip, prepared according to this example, to the same turkey-water suspension, after the suspension had been spiked with E. coli 0157:H7 that had been stored frozen and then thawed. As seen on the plot 300, a much stronger signal was recorded within one minute, as compared with the plot 200 (FIG. 2). Over the course of the experiment, signals exceeding 25 microamperes were recorded, for example at a point 302 and at a point 304. Quantitative bacteriological culture by routine plating of the stock material used for the experiment indicates that the number of colony-forming units (cfu's) in the one milliliter sample tested was approximately 30,000.

Removal of the gold from the black banana lead, electrode 161, resulted in loss of signal, while removal of gold coating from the red banana lead, electrode 160, which was directly in contact with the aluminum foil used as the base member 120, did not cause any change in sensor performance. This result is consistent with the analyte-related charging of a capacitor formed by parallel plates of aluminum foil base member 120, second conductive element 197 gold square, separated by a thin layer of highly insulating nail polish dielectric element 198.

### Second Embodiment

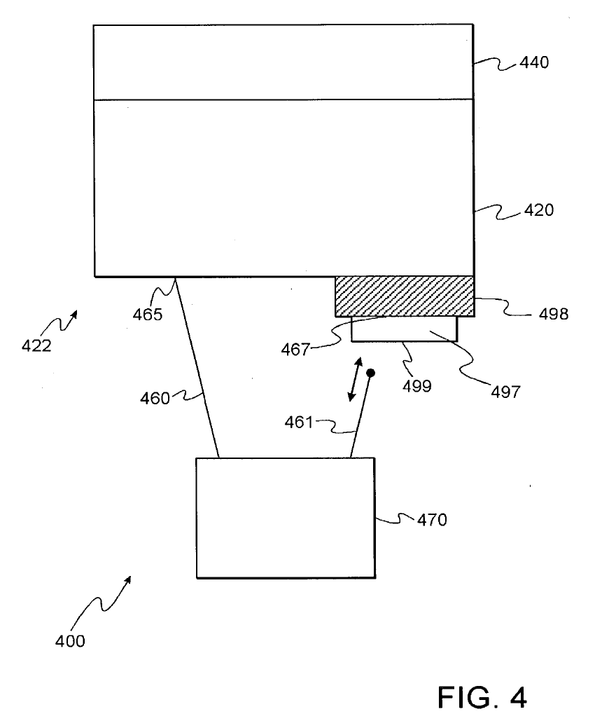

Reference is now made to FIG. 4, which is a schematic of a sensor detection system 400 that is constructed and operative in accordance with an alternate embodiment of the invention. The sensor detection system 400 is similar to the sensor detection system 100 (FIG. 1), and like elements have like reference numerals, advanced by 300. In the sensor detection system 400, the chemical entity 132, the packaging layer 150, and the second conductive element 197 are omitted. A second conductive element 497 is integral with a sensor strip 422, having an area of contact with a dielectric element 498 at a position 467. An electrode 461 is moved into a contacting relationship at a position 499 with the second conductive element 497 during operation, as indicated by the double-pointed arrow in FIG. 4.

### EXAMPLE 2

Using the embodiment of FIG. 4, a conducting polymer is employed as a base member 420. On one side, antibodies for blood-related virus antigens are immobilized to form a binding agent layer 440. The layer is briefly treated with dilute glutaraldehyde to effect partial cross-linking and lattice stabilization. On the opposite side of the base member, Teflon® (Available from E. I. DuPont de Nemours, Inc., 1007 Market Street, Wilmington, Del. 19898) at a thickness of approximately 1-10 microns, is applied to the second conductive element 497 to form the dielectric element 498. The sensor strip 422 is contacted to two electrodes 460, 461 of a digital voltmeter-based detection unit, which is used as a detection unit 470. One of the electrodes 460, 461 is contacted directly to the base member at position 465, while the other one of the electrodes 460, 461 is contacted to the second conductive element 497 at the position 467. A drop of whole blood (not shown) is placed on the sensor strip 422, on the same side as the binding agent layer 440. If a viral antigen is present in the drop of whole blood (not shown), then its interaction with the binding agent layer 440 will lead to a reading or indication in the detection unit 470. The base member 420 and the second conductive element 497 may be of different physical dimensions.

### Third Embodiment

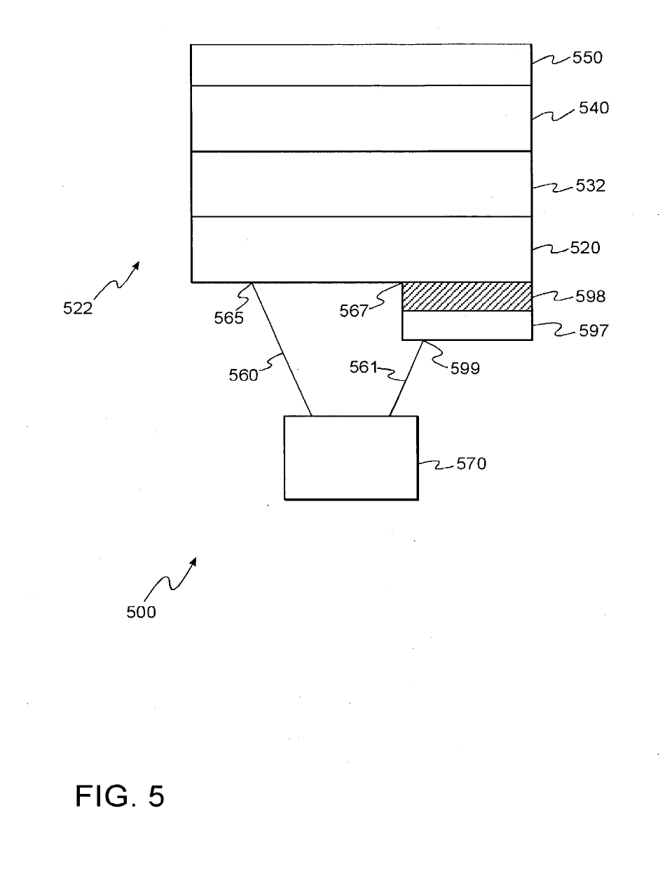

Reference is now made to FIG. 5, which is a schematic of a sensor detection system 500 that is constructed and operative in accordance with an alternate embodiment of the invention. The sensor detection system 500 is similar to the sensor detection system 100 (FIG. 1), and like elements have like reference numerals, advanced by 400. A second conductive element 597 is integral with a detection unit 170, electrode 561 having an area of contact with a dielectric element 598 at a position 567. An electrode 561 is in a contacting relationship at a position 599 with the second conductive element 597. The dielectric element 598 contacts a base member 520 of the sensor strip 522 directly. In this embodiment, the three components of the capacitor, namely the conductive base member 520, the dielectric element 598 and the second conductive element 597 are all physically associated with a disposable sensor strip that includes the binding agent layer 540 and the packaging layer 550.

### EXAMPLE 3

In this example, a metal foil serves as the base member 520. A non-conducting chemical entity 532 is applied to one side of the foil. On the same side of the foil as the chemical entity 532, a binding agent layer 540 is formed above the chemical entity 532 by soaking the coated foil in a solution of single-strand nucleic acid binding agents. A packaging layer 550 is formed above the binding agent layer 540 by soaking the sensor strip 522 in a solution of sodium chloride and sucrose. A detection unit 570 is realized as a digital ammeter, with a first electrode 560, and a second electrode 561, the electrode 561 being coated with a deposited layer to form the second conductive element 597 of silver metal. Above the silver layer is deposited an organic polymer, which serves as the dielectric element 598. The electrode 560 is contacted to the sensor strip 522, directly at the base member at position 565. The second electrode 561 with associated silver and organic dielectric element 598 is similarly contacted to the sensor strip 522 at a position 567. A drop of blood (not shown) is applied to the packaging layer 550, which dissolves to expose the binding agent layer 540. If the analyte DNA single strand is present in the blood, then a current will be generated in a closed sensor circuit 520, 560, 570, 561, 597, 598. In this example, the electrode 561 is coated with silver metal, which serves as the second conductive element 597, while the organic polymer deposited over the indium tin oxide serves as the dielectric element 598

### Fourth Embodiment

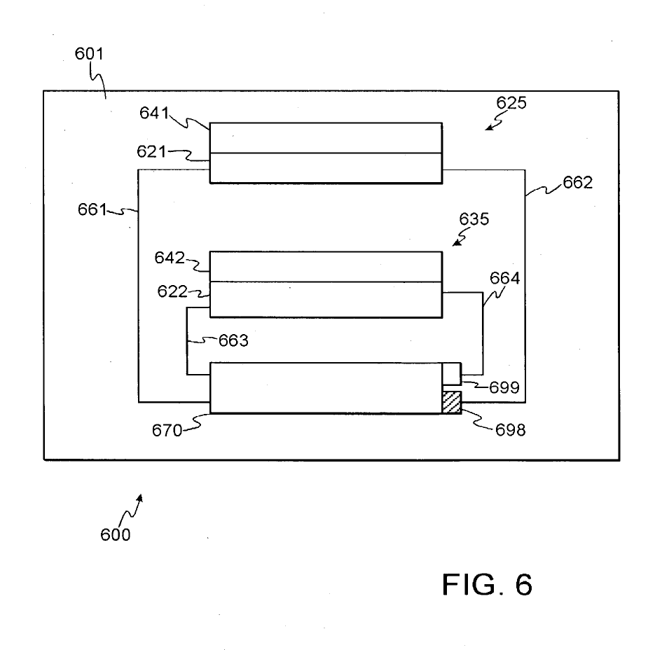

Reference is now made to FIG. 6, which is a schematic of a sensor detection system 600 that is constructed and operative in accordance with an alternate embodiment of the invention. The sensor detection system 600 employs multiplexed sensor strips for multiple analyte detection. A plastic substrate 601, for example polyethylene, is coated with conducting ink lines 621, 622. On each of the conductive ink lines 621, 622 is bound a binding agent layer 641, 642 of a unique antibody specific for a given food pathogenic bacteria, thereby defining two sensing units 625, 635. Each of the ink lines 621, 622 is contacted by two unique leads 661, 662, 663, 664 of a detection unit 670. Two separate dielectric elements 698, 699 are externally located in the detection unit 670, each of the dielectric elements 698, 699 forming a component of a first sensor circuit 670, 698, 662, 625, 661, and a second sensor circuit 670, 699, 664, 635, 663. The operation of the sensing units 625, 635 is similar to the operation of the sensor strip 122 of the sensor detection system 100 (FIG. 1), although the physical arrangement and structure are different.

### EXAMPLE 4

A liquefied food sample is applied to the sensing units 625, 635. The presence of a given pathogenic bacterium causes a generated electrical signal to be recorded in one of the first sensor circuit 670, 698, 662, 625, 661, or the second sensor circuit 670, 699, 664, 635, 663, whichever circuit is associated with the antibody binding agent specific for the given bacterial agent present in the sample.

### Fifth Embodiment

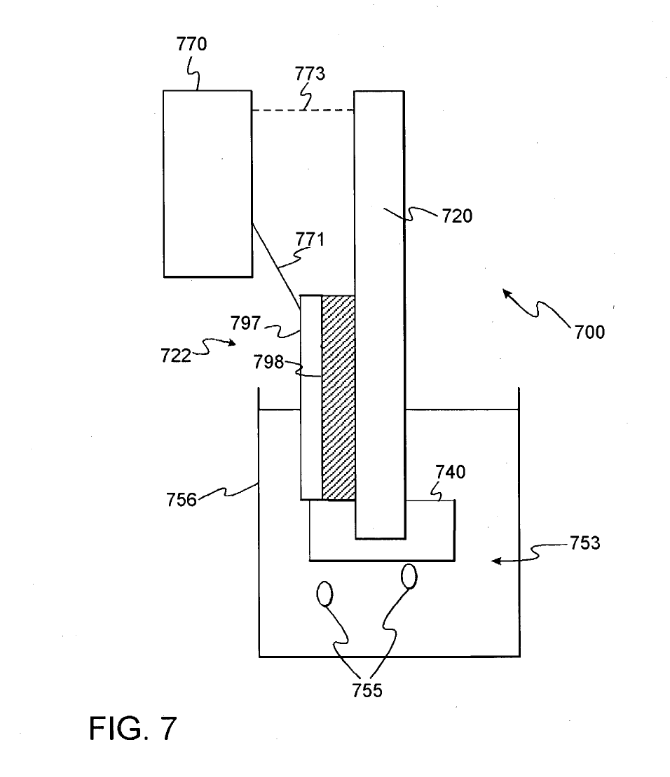

Reference is now made to FIG. 7, which is a schematic of a sensor detection system 700 that is constructed and operative in accordance with an alternate embodiment of the invention. The sensor detection system 700 is similar to the sensor detection system 100 (FIG. 1), wherein like elements have like reference numerals, advanced by 600. A sensor strip 722 is exposed to a sample 753 that contains target analyte 755. Typically, the sample 753 is disposed in a fluid container 756. A detection unit 770 is coupled to the sensor strip 722. The sensor strip 722 includes a base member 720, a binding agent layer 740, a dielectric element 798, a second conductive element 797, and is connected to the detection unit 770 via a pathway 771. In some embodiments the pathway 771 can be realized as an electrode, in which case a second electrode 773, indicated as a broken line in FIG. 7, connects the detection unit 770 to the base member 720. The binding agent layer 740 may contact both sides of the base member 720 as shown in FIG. 7, or may be limited in other embodiments to one side of the base member. The dielectric element 798 sits between and physically separates the base member 720 and the second conductive element 797. There can be no direct physical contact between the base member 720 and the second conductive element 797, as this would result in shorting of the capacitor. The sizes of the components of the sensor strip 722 may vary from microns for chip-based detection systems to centimeters for individual disposable test strips.

Response of the embodiments of the sensor system described herein is very rapid, generally less than 90 seconds. Cost of manufacture is low, and sensitivity has been shown to be sufficiently high for practical analyte detection.

The present invention has been described with a certain degree of particularity, however those versed in the art will readily appreciate that various modifications and alterations may be carried out without departing from the spirit and scope of the following claims. Therefore, the embodiments and examples described here are in no means intended to limit the scope or spirit of the methodology and associated devices related to the present invention.

1. A sensor for detecting an analyte, comprising:
a base member having a conductive electrical property, said base member defining a first conductive element;
a binding agent layer proximate said base member;
a dielectric element proximate said base member; and,
a second conductive element that is physically contacted to said dielectric element and adapted for electrical connection in a circuit with said first conductive element; wherein said base member and said binding agent layer define a sensor strip.

2. The sensor according to claim 1, further comprising a chemical entity bound to said base member and disposed between said base member and said binding agent layer.

3. The sensor according to claim 1, wherein said binding agent layer contacts two surfaces of said base member.

4. The sensor according to claim 1, further comprising two equipotential leads coupling said sensor strip to a detection unit, wherein at least one of said equipotential leads is electrically contacted to said second conductive element.

5. The sensor according to claim 1, further comprising a packaging layer disposed above said binding agent layer, said packaging layer being soluble in a medium that contains the analyte.

6. The sensor according to claim 1, wherein said dielectric element is an organic compound and is physically associated with said base member on a first side of said dielectric element, and with said second conductive element on a second side of said dielectric element.

7. The sensor according to claim 1, wherein said sensor strip comprises a plurality of sensor strips.

8. A method for detecting a predetermined analyte, comprising the steps of:
providing an electrically conductive base member, said base member defining a first conductive element;
forming a binding agent layer of macromolecules in proximity to said base member, wherein said macromolecules are capable of interacting at a level of specificity with said predetermined analyte,
disposing a dielectric element proximate said base member, wherein said base member, said binding agent layer and said dielectric element define a sensor strip;
exposing said predetermined analyte to said binding agent layer; and,
detecting an electrical current generated in a closed electrical circuit, said electrical current being responsive to a presence of said predetermined analyte, wherein said closed electrical circuit comprises said base member, said dielectric element, and a second conductive element physically contacted to said dielectric element.

9. The method according to claim 8, further comprising the steps of:
binding a chemical entity to said base member; and
forming said binding agent layer proximate said chemical entity.

10. The method according to claim 8, wherein said step of detecting is performed by equipotentially coupling leads of a detection unit to said sensor strip, wherein one of said leads is coupled to said second conductive element.

11. The method according to claim 10, wherein said step of coupling said detection unit is performed by contacting electrodes to said sensor strip, electrical passivity of said electrodes being maintained while performing said step of coupling.

12. The method claim 8, wherein said second conductive element is physically associated with said sensor strip.

13. The method according to claim 8, wherein said dielectric element is an electrically insulating material and is physically associated with said base member on a first side of said dielectric material, wherein said second conductive element is physically associated with a second side of said dielectric material.

14. The method according to claim 8, further comprising the step of disposing a packaging layer above said binding agent layer, said packaging layer being soluble in a medium that contains said predetermined analyte.

15. The method according to claim 8, wherein said sensor strip comprises a plurality of sensor strips.

16. A method for detecting a predetermined analyte, comprising the steps of:
providing an electrically conductive base member;
forming a binding agent layer of macromolecules in proximity to said base member, wherein said macromolecules are capable of interacting at a level of specificity with said predetermined analyte,
forming a capacitor on said base member, wherein said base member comprises an element of said capacitor, said capacitor and said base member defining a sensor strip;
exposing said predetermined analyte to said binding agent layer; and,
detecting an electrical current generated in a closed electrical circuit, said electrical current being responsive to a presence of said predetermined analyte, wherein said closed electrical circuit comprises said sensor strip.

17. The method according to claim 16, further comprising the steps of:
binding a chemical entity to said base member; and
forming said binding agent layer proximate said chemical entity.

18. The method according to claim 16, wherein said step of detecting is performed by equipotentially coupling leads of a detection unit in said closed electrical circuit, wherein one of said leads is coupled to said capacitor.

19. The method according to claim 18, wherein said step of coupling leads is performed while maintaining electrical passivity of said leads.

20. The method according to claim 16, further comprising the step of disposing a packaging layer above said binding agent layer, said packaging layer being soluble in a medium that contains said predetermined analyte.
